(19) United States
(12) Patent Application Publication
(10) Pub. No.: US 2024/0115638 A1
(43) Pub. Date: Apr. 11, 2024

(54) APPARATUS, SYSTEM AND METHODS FOR PROCESSING OF HARVESTED CANNABIS PLANT MATERIAL

(71) Applicant: EQALIS GROUP NEW ZEALAND LIMITED, Hamilton (NZ)

(72) Inventor: Greg Charles MISSON, Hamilton (NZ)

(21) Appl. No.: 18/259,117

(22) PCT Filed: Dec. 24, 2021
(86) PCT No.: PCT/NZ2021/050235

§ 371 Date: Jun. 23, 2023

(30) Foreign Application Priority Data
Dec. 24, 2020 (NZ) 771591
Dec. 24, 2020 (NZ) 771593
Mar. 30, 2021 (NZ) 774531
Dec. 23, 2021 (NZ) 783924

(51) Int. Cl. A61K 36/185; B01D 11/02; B01D 29/23; B01D 33/06; B01D 33/15; B01D 33/48; B01D 36/02; B02C 23/36; B02C 23/40

(52) U.S. Cl. CPC A61K 36/185; B01D 11/028; B01D 11/0288; B01D 29/23; B01D 33/06; B01D 33/15; B01D 33/48; B01D 36/02; B02C 23/36; B02C 23/40; A61K 2236/15; A61K 2236/39; B02C 18/10

(57) ABSTRACT

The present technology is directed towards providing an apparatus, system and method for the cost-effective extraction of compounds of interest, such as CBD and THC, from freshly harvested cannabis plant material. The system includes a processing module with a tank that has a flow path to a separator. The tanks are filled with a cooling agent and cannabis plant material which is immersed and agitated to break up the plant material. The filtered outflow from the tank is a filtrate of a mixture of cooling agent and broken up plant material that is transferred to the separator to separate out at least some trichomes containing the compounds of interest.

### CROSS-REFERENCE TO RELATED APPLICATIONS

This application is a national phase application under 35 U.S.C. § 371 of International Application No. PCT/NZ2021/050235 filed Dec. 24, 2021 entitled, “APPARATUS, SYSTEM AND METHODS FOR PROCESSING OF HARVESTED CANNABIS PLANT MATERIAL,” which claims the benefit of and priority to New Zealand Patent Application No. 771591 filed Dec. 24, 2020; New Zealand Patent Application No. 771593 filed 24 Dec. 2020; New Zealand Patent Application No. 774531 filed Mar. 30, 2021; and New Zealand Patent Application No. 783924 filed Dec. 23, 2021, the contents of each of which being incorporated by reference in their entireties herein.

### TECHNICAL FIELD

The present disclosure relates to an apparatus, system, and method for the processing of harvested cannabis plant material. The embodiments of the present disclosure have particular application to the extraction of target compounds of cannabinoids, such as cannabidiol or tetrahydrocannabinol and Varian analogues, from freshly harvested cannabis.

### BACKGROUND

Cannabis, primarily Cannabis sativa but also Cannabis indica and Cannabis ruderalis, for medicinal purposes such as pain relief, is legally recognised in many countries, including the United States and New Zealand. It can be provided to the consumer as a dried product (leaves or flowers) or as a dosage in a conventional pharmaceutical form, such as a pill, tablet, or as an oil/liquid for oral consumption. There is also an increasing need to separate specific compounds, in certain forms, from cannabis plant material for use in medicaments for treatment of specific health conditions.

Consequently, cannabis plants are being cultivated on an increasing scale to meet demand. However, cultivating cannabis plants on a commercial scale has a number of issues, due to space constraints, availability of resources, and technological inefficiencies. This means the cost of purchasing medicinal cannabis can be significant, a reflection of the expense involved in growing, processing and extracting from the cannabis plant the valuable components, the cannabinoids, such as cannabidiol (CBD) or tetrahydrocannabinol (THC), into a form that is vendible to the consumer.

This expense can make it difficult for persons requiring medicinal cannabis to access the product. It can also make it prohibitively expensive for potential cannabis farmers to enter the market, which in turn keeps the price of cannabinoid containing medicines high.

To optimise growth of the cannabis plant, they are often grown indoors and most growers supplying medicinal cannabis to the market take this approach. While this allows the grower to better control the temperature, light conditions, and security of the plant crop, a drawback is that a major amount of infrastructure is required, from growing space, i.e., buildings, to electrical equipment and watering paraphernalia.

Once harvested, the cannabis plant material degrades relatively quickly and is vulnerable to mold and pestilent insects. The harvested cannabis plant material also has a significant moisture content, which can be as much as 70% and contributes to its undesirable breakdown. This moisture needs to be eliminated or at the very least significantly reduced before the plant material can be further processed. The drying step is both labour and energy demanding, as the plants need to be trimmed and stored in a relatively dehumidified environment for drying for up to two to three weeks.

Harvesting of the cannabis plants usually involves removal of a major proportion of their biomass, the stems and fan leaves, leaving only the sugar leaves and flowers. These contain the valuable component of interest, a structure known as a trichome, a bulbous resinous gland on a thin stem. It is the trichomes which contain the target compounds, cannabinoids such as CBD and THC, that need to be extracted with further processing.

A common commercial approach to extracting target compounds/value components from cannabis plant material involves a multi-stage process. First, the moisture level in the plant matter containing the trichomes is reduced to only around 10% of the overall biomass of the plant matter. The cannabinoids of value can then be extracted, which usually entails the application of solvents, such as ethanol, to the trichomes. However, this requires the use of complex scientific equipment, with skilled staff being necessary to operate the processing machinery and who need to be remunerated appropriately. The solvents used in the processing can be hazardous and must be stored in safe, secure environments and also disposed of appropriately.

Another approach to separating out the trichomes from the plant matter involves immersing it in a tank of cool or chilled water and gently agitating the resulting mixture. This separates the trichomes from the rest of the plant matter and they can then be captured and extracted from the water using a separator. An example of such an arrangement is disclosed in PCT Application No. PCT/CA2018/051197. However, a drawback to this arrangement is that it can be very wasteful of water, particularly on a commercial scale, as it typically requires 20 parts of water to one part plant matter for optimum operation. After the separation of the trichomes from the water, it is then discarded as waste. Furthermore, the separation of the trichomes from the plant matter can also be relatively prolonged and time inefficient.

As will be appreciated there is considerable capital and operational expense in growing and processing cannabis plants for the purpose of supplying medicinal cannabis. Together with the time and processing inefficiencies of existing extraction processes, this has a bearing on the retail price for the end product. In some markets, this may mean medicinal cannabis or medicines containing cannabinoids may be financially inaccessible to at least some of those individuals who need it for treatment of health problems and/or pain relief.

A solution to the aforementioned problems is desired.

### BRIEF SUMMARY

The present technology is directed towards providing an apparatus, system and method for the cost-effective extraction of compounds of interest, i.e. value components, from freshly harvested cannabis plant material. These compounds of interest include, but are not limited to, CBD, THC and Varian analogues thereof.

In particular, the present technology provides an apparatus, system and method for the removal of the value components of the cannabis plant material, the trichomes. These are the parts of the plant that are particularly high in CBD and THC compounds at certain stages of the life of the plant.

First Aspect

According to one aspect of the present technology, there is a provided a method of separating trichomes from harvested cannabis plant material, wherein the method includes the steps of:
a) charging a first vessel with a quantity of harvested cannabis plant material and a quantity of a cooling agent to create a first mixture;
b) filtering and/or agitating the first mixture in the first vessel to produce a first laden quantity of cooling agent containing a first filtrate including at least some trichomes;
c) withdrawing the first laden quantity of cooling agent from the first vessel and transferring same to a first separator;
d) operating the first separator to remove at least a portion of the trichomes from the first filtrate of the first laden quantity of cooling agent;
e) transferring at least a portion of the first laden quantity of cooling agent to a second vessel to create a second mixture;
f) filtering and/or agitating the second mixture in the second vessel to produce a second laden quantity of cooling agent containing a second filtrate including at least some trichomes;
g) withdrawing the second laden quantity of cooling agent from the second vessel and transferring to a second separator;
h) operating the second separator to remove at least a portion of trichomes from the second filtrate of the second laden quantity of cooling agent,

the method characterised in that step e) is performed concurrently with step c).

According to a further aspect of the present technology, the method includes an additional step of:
i) transferring at least a portion of the second laden quantity of cooling agent to a third vessel create a third mixture, wherein step i) is performed concurrently with step g).

Optionally, the method is performed using a system having a first processing module and a second processing module, wherein the first processing module includes a first separator and a first vessel, and wherein the second processing module includes a second separator and a second vessel.

Optionally, each of the first and second processing modules include an agitator disposed within the vessel to vigorously agitate and mechanically work the mixture of the cooling agent and quantity of harvested cannabis plant material prior to or concurrently with steps b) and f). Optionally, at least one of the first and second processing modules include a further agitator for agitation of the laden quantities of cooling agent during the transfer of same to the separators of the respective first and second processing modules. Optionally, each of the first and second processing modules include a pump, and during steps c) and g), the first and second laden quantities of cooling agent containing the first and second filtrates respectively are passed through the pump to agitate same prior to being introduced to the first and second separator respectively.

Alternatively or additionally, each of the first and second processing modules include a static mixer, and during steps c) and g), the first and second laden quantities of cooling agent containing the first and second filtrates respectively are passed through the static mixer and a first portion of the first and second laden quantities of cooling agent is returned to the first and second vessel respectively and a second portion of same is introduced to the first and second separator respectively.

Optionally, at least 20% to 70% of the first and second laden quantity of cooling agent is returned to the first and second vessel respectively after passing through the static mixer.

Alternatively or additionally, each of the first and second processing modules include a scrape surface filter and, during steps c) and g), the first and second laden quantity of cooling agent is passed through the scrape surface filter and a first portion of the first and second laden quantities of cooling agent is diverted back to the first and second vessel respectively and a second portion of same is introduced to the first and second separator respectively.

Optionally, at least 20% to 70% of the first and second laden quantity of cooling agent is returned to the first and second vessel respectively after passing through the scrape surface mixer, with 30% to 80% proceeding to the first and second separator respectively.

Optionally, at least 30% to 80% of the first and second laden quantity of cooling agent is returned to the first and second vessel respectively after passing through the first and second separator respectively.

Optionally, during step e), the cooling agent is chilled before being introduced to the second vessel.

Optionally, the cooling agent is a slurry of ice and water. Alternatively, the cooling agent is chilled water.

Optionally the cooling agent is chilled to a temperature of between 0° Celsius to 5° Celsius. Even more optionally, the cooling agent is chilled to a temperature substantially at or close to 0° Celsius.

Optionally, the ratio of cooling agent to plant material is from between 0.5:1 to 10:1. Even more optionally, the ratio of cooling agent to plant material is between 1:1 to 5:1. In a particular embodiment, the ratio of cooling agent to plant material is approximately 2:1.

Optionally, step a) is performed over a period of 15 to 45 minutes. Even more optionally, step a) is performed over a period of approximately 30 minutes.

Optionally, step b) is performed for between 30 minutes to six hours. Even more optionally, step b) is performed for approximately 90 minutes.

Optionally, steps a) to d) are performed for between one hour to eight hours. Even more, optionally, steps a) to d) are performed for approximately 150 minutes.

Optionally, during step e) the transfer of the portion of the first laden quantity of cooling agent to the second vessel occurs continuously.

Optionally, the portion of the first laden quantity of cooling agent transferred to the second vessel initially serves to charge the second vessel.

Optionally, during step i) the transfer of the portion of the second laden quantity of cooling agent to the third vessel occurs continuously.

Optionally, the portion of the second laden quantity of cooling agent transferred to the third vessel initially serves to charge the third vessel.

Optionally, prior to step a), the harvested cannabis undergoes a pre-processing step where the stalks and/or fan leaves are substantially removed or reduced in size through the use of cutting blades or the like.

According to a further aspect of the present technology, there is provided a system for use in the method of separating trichomes from harvested cannabis plant material substantially as described above, wherein the system includes:
a first processing module and a second processing module, wherein each module includes:
   a separator; and
   a vessel, wherein the vessel is configured to receive a cooling agent and is provided with:
      a filtering medium; and
      an outlet, wherein the outlet is communicative with the separator and arranged to transfer a quantity of cooling agent laden with harvested cannabis plant material to the separator;
wherein the system includes a transfer mechanism configured to transfer at least a portion of the cooling agent received by the vessel of the first processing module to the vessel of the second processing module.

Optionally, the vessel of each processing module is an open tank. Even more optionally, the vessel is a substantially cylindrical open tank.

Optionally, the tank has a continuous side wall and a base.

Optionally, the outlet of the tank is at or proximate the base.

Optionally, the tank of the first processing module includes a charger for the cooling agent to be introduced to the tank.

Optionally, the tank of the first processing module includes a charger for the harvested cannabis plant material to be introduced to the tank.

Optionally, the filtering medium of each tank is a substantially cylindrical basket.

Optionally, the dimensions of the cylindrical basket are 80% to 95% of the dimensions of the tank.

Optionally, the cylindrical basket includes a continuous side wall with a plurality of perforations. Even more optionally, the continuous side wall is fabricated as a mesh.

Alternatively, the filtering medium is a screen at or proximate the outlet of the tank.

Optionally, the tank includes an agitator. Even more optionally, the agitator is arranged to agitate the contents of the filtering medium when in use.

Optionally, each module includes a flow path for the cooling agent laden with harvested cannabis plant material from the outlet to the separator. Even more optionally, each module includes a pipeline from the outlet to the separator.

Optionally, each module includes a pump on the pipeline between the outlet and the separator. Even more optionally, the pump is configured to agitate the cooling agent prior to being introduced to the separator.

Optionally, each module includes a static mixer on the pipeline between the outlet and the separator. Even more optionally, the static mixer is configured to agitate the cooling agent prior to being introduced to the separator.

Optionally, each module includes a scrape surface filter on the pipeline between the outlet and the separator. Even more optionally, the scrape surface filter is on the pipeline between the pump/static mixer and the separator.

Optionally, the separator is a rotary mechanical or fixed filter separation device with a filter of between 1 to 200 microns. Alternatively, the separator is a singular or plurality of cyclonic separators.

Optionally, each module includes one or more recirculation loops configured to, in use, return at least a portion of the cooling agent from the separator to the vessel.

Optionally, each module includes a first recirculation loop, wherein the first recirculation loop is a pipeline for transferring at least some cooling agent from the scrape surface filter back to the tank.

Optionally, the first circulation loop includes a cooling means to bring the cooling agent to a desired temperature before it is returned to the tank.

Optionally, each module includes a second recirculation loop, wherein the second recirculation loop is a pipeline for transferring at least some cooling agent from the separator back to the tank.

Optionally, the second circulation loop includes a cooling means to bring the cooling agent to a desired temperature before it is returned to the tank.

Optionally, each module includes a pipeline from the separator for transferring separated filtrate for further processing. Even more optionally, the pipeline of each separator drains to a common line for transferring separated filtrate for further processing.

Optionally, the transfer mechanism includes a conduit.

Optionally, the conduit of the transfer mechanism includes an inlet and an outlet.

Optionally, the inlet of the conduit is located before the separator of the first processing module. Even more optionally, the inlet of the conduit is located after one of the pump, static mixer or scrape surface filter of the first processing module.

Optionally, the outlet of the conduit is located at or proximate the tank of the second processing module. Even more optionally, the outlet of the conduit is the charger for the tank of the second processing module.

In some embodiments, the tank of the second processing module includes a supplementary charger for further cooling agent and/or harvested cannabis plant material.

It will be appreciated that the present system and method provides for the continual processing of separating trichomes from harvested cannabis plant material.

At the start of operation of the system, cooling agent and harvested cannabis plant material is provided to the vessel of the first processing module and optionally the second and any subsequent processing modules are empty. Once the vessel of the first processing module reaches its operational capacity, the introduction of cooling agent and harvested cannabis plant material still continues, creating an overflow.

A portion of this overflow, cooling agent that is laden with harvested cannabis plant material that has been agitated and broken up into shreds and particulate matter, is diverted to the separator while the remaining overflow is the first laden quantity of cooling agent which is transferred to the vessel of the second processing module.

This serves to charge the vessel of the second processing module until it reaches operating capacity. Once this is achieved, the first laden quantity of cooling agent still continues to flow in the vessel of the second processing module creating an overflow; as with the first processing module, a portion is diverted to the separator and the remainder forms the second laden quantity of cooling agent is which transferred to the vessel of the third processing module.

In this manner, each vessel of the respective processing modules reaches operational capacity in turn, beginning with that of the first processing module. Once the system is fully operational, i.e. all the vessels of the processing modules that are present have reached capacity, the amount of laden cooling agent provided to the vessel of the second and subsequent processing modules is substantially the same as that transferred to the vessel of the following processing module less the portion delivered to the separator. In some instances, the laden cooling agent may be supplemented with cooling agent to substantially compensate for the proportion that has been diverted to the separator.

When the system ceases operation, the supply of cooling agent and harvested cannabis plant material to the vessel of the first processing module ceases. The system will then substantially be emptied, beginning with the first processing module; the first laden quantity of cooling agent continues to be transferred to the vessel of the second processing module and will do so until the vessel of the first processing module is substantially empty. In the meantime, there is still transfer of the second laden quantity of cooling agent to the vessel of the third processing module. This will continue until the vessel of the second processing module is substantially empty.

In alternative embodiments, each vessel of each processing module may be charged with cooling agent and harvested cannabis plant material before the system becomes operational. However, once the system becomes operational, continual introduction of cooling agent and harvested cannabis plant material may only be to the first processing module.

Second Aspect

According to one aspect of the present disclosure, there is provided a system for use in a method of separating trichomes from harvested cannabis plant material, wherein the system includes:
a processing module that includes:
   a vessel, wherein the vessel is configured to receive a cooling agent and harvested cannabis plant material and wherein the vessel is provided with an outlet;
   an agitator configured to agitate the harvested cannabis plant material in the vessel;
   a first separator and a second separator;
   the processing module including a first flow path from the outlet of the vessel to the first separator, the flow path arranged to transfer a quantity of cooling agent laden with agitated harvested cannabis plant material to the first separator; and
   a second flow path from the first separator to a second separator, the second flow path arranged to transfer a first stream of material derived from the quantity of cooling agent laden with agitated harvested cannabis plant material from the first separator to the second separator.

Optionally, the vessel of the processing module is an open tank. Even more optionally, the vessel is a substantially cylindrical open tank.

Optionally, the vessel has a continuous side wall and a base.

Optionally, the outlet of the vessel is at or proximate the base.

Optionally, the processing module includes a filtering container, wherein the filtering container is configured to be received within the vessel and receive harvested cannabis plant material.

Optionally, the filtering container is a substantially cylindrical basket with a continuous side wall including a plurality of perforations and a base. Even more optionally, the side wall is fabricated as a mesh.

Optionally, the dimensions of the filtering container are 80% to 95% of the dimensions of the vessel,

Optionally, the processing module includes a charger which is configured to introduce the cooling agent to the vessel.

Optionally, the processing module includes a charger for the harvested cannabis plant material which is configured to introduce the harvested cannabis plant material to at least one of the vessel and the filtering container.

Optionally, the charger for the harvested cannabis plant material includes a mill to reduce the size of the harvested cannabis plant material passing therethrough.

Optionally, the mill is located and arranged to feed milled harvested cannabis plant material into at least one of the vessel or the filter container.

Optionally, the agitator is configured to mechanically work and break up the harvested cannabis plant material into shreds and/or particulate matter and work it into a mixture or slurry with the cooling agent.

Optionally, the agitator includes a plurality of blades and tines to mechanically work and break up the harvested cannabis plant material into shreds and/or particulate matter.

Optionally, the flow path from the outlet of the vessel to the first separator is a first pipeline.

Optionally, the flow path from the first separator to the second separator is a second pipeline.

Optionally, the respective flow paths include a pump. Even more optionally, the pump is configured to agitate the mixture of cooling agent and harvested cannabis plant material prior to being introduced to the first and/or second separator.

Optionally, the processing module is configured to deliver an amount of cooling agent and harvested cannabis plant material to the vessel and/or filtering container in an amount and at a rate substantially the same as the amount and rate at which the slurry of cooling agent and harvested cannabis plant material is delivered to the first and/or second separator.

Optionally, the first separator includes a rotating screen having a first end and second end.

Optionally, the mixture of cooling agent and harvested cannabis plant material is delivered to the rotating screen at the first end.

Optionally, the first separator includes a conveyor at or proximate the second end of the rotating screen, the conveyor receiving the mixture of cooling agent and harvested cannabis plant material after it has passed through the rotating screen.

Optionally, the conveyor is a helical conveyor. Even more optionally, the conveyor is an auger. In alternative embodiments, the conveyor may be a belt or series of plates with a press or rollers along part of its length.

Optionally, the conveyor is inclined and has an upper end and a lower end.

Optionally, the conveyor has an upper outlet at its upper end and a lower outlet at or proximate its lower end.

Optionally, the upper outlet of the conveyor is an exit point for harvested cannabis plant material passing through the conveyor. It will be understood that the harvested cannabis plant material is largely spent, having at least the majority of the trichomes removed therefrom.

Optionally, the lower outlet of the conveyor is an exit point for a first stream of material derived from the mixture of cooling agent and harvested cannabis plant material. The first stream should be understood to include trichomes and cooling agent.

Optionally, the lower outlet of the auger is communicative with the flow path to the second separator.

Optionally, the first separator includes one or more rinse down points proximate the rotating screen.

Optionally, the first separator includes a screen to receive a rinse agent.

Optionally, the first separator includes a flow path from the screen receiving the rinse agent to the second separator.

Optionally, the second separator is a rotary drum screen.

Optionally, the second separator includes a first flow path for the trichomes and a second flow path for cooling agent. In a particular embodiment, the second flow path leads to the wash down points for the first separator. In alternative embodiments the second flow path may also, or alternatively, return to the vessel of the processing module.

According to a further aspect of the present disclosure, there is provided a method of using a system substantially as described above, the method including the steps of:
a) filling the vessel with a cooling agent and harvested cannabis plant material;
b) agitating the mixture of harvested cannabis plant material and cooling agent to form a mixture;
c) transferring a quantity of the mixture of cooling agent and harvested cannabis plant material to the first separator;
d) separating at least a portion of the mixture of cooling agent and harvested cannabis plant material into a first stream containing trichomes and cooling agent and a second stream containing harvested cannabis plant material;
e) transferring the first stream to the second separator.

According to a further aspect of the present technology, the method includes an additional step of:
f) using the second separator to separator the first stream into a high value stream of trichomes and a stream of spent cooling agent.

Optionally, the cooling agent used in step a) is a mixture of ice and water. Alternatively, the cooling agent is chilled water.

Optionally the cooling agent is chilled to a temperature of between 0° Celsius to 5° Celsius. Even more optionally, the cooling agent is chilled to a temperature substantially at or close to 0° Celsius.

In some embodiments, during step a), the harvested cannabis plant material is placed into a filtering container which in turn is placed into the vessel.

Optionally, during step a) the cooling agent and harvested cannabis plant material is added to the vessel in an amount and at a rate substantially corresponding to the amount and rate at which the slurry of cooling agent and harvested cannabis plant material is transferred to the first separator during step c).

Optionally, during step b) the agitator mechanically works and breaks down the harvested cannabis plant material into shredded and particulate plant matter and trichomes.

Optionally, during step b) the shredded cannabis plant material and trichome are worked into a mixture or slurry with the cooling agent.

Optionally, during step c), as the quantity of the mixture of cooling agent and harvested cannabis plant material is transferred to the first separator, a similar quantity of cooling agent and harvested cannabis plant material is added to the vessel.

In some embodiments, during step c), at least some of the harvested cannabis plant material is filtered and prevented from being transferred to the first separator.

Optionally, during step d), the agitated harvested cannabis plant material is rinsed by a rinse agent as it passes through the first separator.

Optionally, at least some of the rinse agent is reclaimed cooling agent, e.g., that has been reclaimed from agitated harvested cannabis plant material passing through the second separator during step f).

Optionally, the method includes the step of cooling the reclaimed cooling agent prior to being used as a rinse agent.

Optionally, the method includes the step of collecting the rinse agent, and more optionally separating any trichomes contained therein from the rinse agent.

Optionally, the method includes the step of milling the harvested cannabis plant material prior to introducing same to the vessel and/or filtering container.

It will be appreciated that the present system and method provides for the processing of separating trichomes from harvested cannabis plant material. It is particularly helpful when used in a continuous process as the system can be operated in way to ensure a continuous inwards feed of harvested cannabis plant material and cooling agent at a rate and quantity substantially matching the output from the separators. This helps with efficiencies and economy of operation.

### BRIEF DESCRIPTION OF DRAWINGS

One of more embodiments of the present technology will be described below by of example only, and without intending to be limited, with reference to the following drawings, in which like reference numerals refer to similar elements including:

FIG. 1 is a flow chart for the steps of processing harvested cannabis;

FIG. 2 is a schematic of a system for processing harvested cannabis in accordance with an aspect of the present technology;

FIG. 3 shows a further schematic of the system of FIG. 2;

FIG. 4 shows a schematic of one example of a processing module for a system for processing harvested cannabis in accordance with an aspect of the present technology;

FIG. 5 shows a schematic of the processing module of FIG. 4 in operation with a pathway including a first recirculation loop to the vessel of the system for processing harvested cannabis;

FIG. 6 shows a schematic of the processing module of FIG. 4 in operation with a pathway including a second recirculation loop to the vessel of the system for processing harvested cannabis;

FIG. 7 shows a schematic of the module of FIG. 4 in operation with a pathway to the separator of the system for processing harvested cannabis;

FIG. 8 shows a schematic of the processing module of FIG. 4 in operation with a pathway to a second module of the system for processing harvested cannabis;

FIG. 9 shows a schematic of a system for processing harvested cannabis that includes a plurality of processing modules in continuous operation;

FIG. 10 shows a schematic of another example of a processing module for a system for processing harvested cannabis in accordance with an aspect of the present technology; and

FIG. 11 shows a schematic of the separators of the processing module of FIG. 10 in accordance with an aspect of the present technology.

### DETAILED DESCRIPTION

1.1. Processing Method for Freshly Harvested Cannabis Plant Material

Embodiments of the present technology relates to methods and equipment used to process harvested cannabis.

Harvested cannabis should be understood to mean cannabis plant material that has reached an age appropriate for processing, which is typically eight to ten weeks after flowering. The cannabis plant comprises three main parts; the stalk, the leaves (fan leaves, which are the larger, sun-collecting leaves of the plant, and the sugar leaves, that form around the flower, or bud, of the plant), and the flower.

The value components of the cannabis plant are substantially contained in the trichomes. These are found primarily in the sugar leaves and the female flowers of the plant. The trichomes are not found in major quantities in the male flowers.

The colour of the trichomes may be indicative of when the plant should be harvested; if clear, there is minimal THC and/or CBD present; if somewhat translucent or cloudy, an increasing amount of THC/CBD is present. If amber, the THC/CND concentration declines. The grower will optionally harvest the cannabis plants when the THC/CBD presence in the trichomes is optimal.

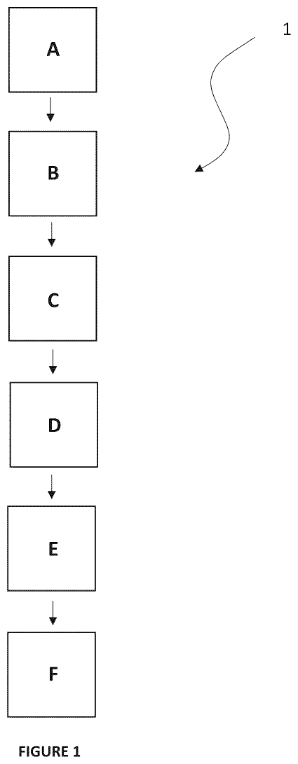

One method of harvesting essentially involves separating the stalk from the root structure using cutting equipment. Following harvesting, the resulting plant material goes through several steps of processing, generally indicated as arrow 1, as indicated in FIG. 1: A) pre-processing; B) pre-concentration; C) clarification; D) de-watering; E) extraction; and F) refining.

Each of these steps shall now be described.

5.1.1 Pre-Processing

Once harvested, the cannabis plant material undergoes an initial pre-processing step A, which involves the removal of the larger, lower value/non-productive components of the plant, such as the stalks and fan leaves. This leaves the valuable components of the plant material, the buds/flower and sugar leaves for further processing in accordance with the present technology.

In one example, the pre-processing step involves the de-stalking of the plant material. The stalks of the cannabis plant are of comparatively low value. Alternatively, the value components of the plant material may be harvested in the growing area, leaving the stalks and/or fan leaves behind.

In one example, the pre-processing step involves introducing the plant material to a cutting and/or milling machine. Optionally, during this process, the plant material is reduced to dimensions of around 1 mm to 50 mm. These dimensions may be dependent on the technology involved in the following steps of the processing method and the genetic traits of the specific plants being processed. For example, in some instances, the cutting and/or milling may reduce the dimensions of the plant material to around 1 mm to 25 mm.

In some examples of the present technology, the plant material may be cooled and/or dehumidified for a short period of time during or immediately following the pre-processing step A. In one example, the cooling may be achieved through the introduction of chilled water to the cutting and/or milling machine concurrently with the plant material. In another example, chilled air may be directed onto the plant material as it is conveyed to the cutting and/or milling machine.

5.1.2 Pre-Concentration

Following pre-processing, the remaining plant material, the flowers and sugar leaves, are concentrated and then further separated into a filtrate stream and a waste stream in a pre-concentration step B.

The filtrate stream should be understood to contain the overwhelming majority of the value components, i.e. cannabinoids, while the waste stream should be understood to be the low value-by product resulting from the pre-concentration step. For example, the waste stream comprises the larger particulate plant matter, such as leaves and residual stalks and stems. This waste is removed from processing at the completion of the pre-concentration step B.

A notable aspect of the pre-concentration step B is the introduction of the pre-processed cannabis plant material to a quantity of cooling agent and then agitating the mixture. This agitation is particularly aggressive and mechanically works the immersed cannabis plant material. This helps to expeditiously separate the value components, particularly the trichomes, from the plant material. It may also help to break up the trichomes into smaller fragments and components. When break up of the trichomes occurs, this releases the cannabinoids, in oil form, into the cooling agent.

The term cooling agent should be understood as meaning a fluid (e.g., a liquid or a gas) which reduces the temperature of the plant material when they come into contact with each other. In one form, the cooling agent is a slurry of chilled water and particulate ice, in the form of cubes or chunks, that has been reduced in temperature to substantially 0° Celsius, or close to 0° Celsius. This is advantageous for when trichomes are exposed to temperatures close to freezing, they become brittle and are more easily separated from the flowers and leaves of the plant material with appropriate agitation action.

In another example, the cooling agent may be brine (salt water) with particulate ice. This may be useful since brine can be used in following steps for rinsing the filtrate. Its use as a cooling agent could optimise the efficiency of the processing of the filtrate. It also allows for the further reduction of the temperature of the cooling agent since brine has a lower freezing point than fresh water.

In further examples, the cooling agent may include additives for the purpose of improving efficiencies in the processing of the filtrate. In one such example, a pH adjuster, such as salt, may be introduced to the slurry of ice and water. In another such example, a surfactant may be added to the cooling agent; this may assist in the separation of the trichomes from the remaining plant matter and/or cooling agent.

This reduction in temperature of the harvested plant material increases the efficiency of the pre-concentration phase and the separation of the plant material being processed into filtrate and waste streams. In one example, the filtrate contains around 10% or less of the plant material, much of which is the trichomes, that is pre-concentrated with the remaining 90% or so forming the waste stream.

The quantity of cooling agent used in the pre-concentration step B may depend on the type used and the amount of plant material to be processed at any one time. In one form, when the cooling agent is a slurry of chilled water and ice, the ratio of cooling agent to plant material is from 0.5:1 to 10:1 or, optionally, 1:1 to 5:1. In particular embodiments, the ratio of cooling agent to plant material is approximately 2:1.

The present technology relates to a system, apparatus and method of using same to perform the pre-concentration step, and specifically the removal of certain parts of the harvested cannabis plant material, specifically the trichomes and their constituent components, and processing these into a filtrate stream and a waste stream.

It should be appreciated that in some examples, the waste stream may still contain some trichomes; in these examples, at least a portion of the waste stream may be recirculated back into the system via a number of pathways and the method repeated to extract at least some of these trichomes. This is a notable aspect of the disclosure as it helps make the overall processing of harvested cannabis plant material more efficient than conventional systems, while optimising the use of the cooling agent, thereby minimising wastage. This provides a method and system for processing of harvested cannabis at volumes that are more economically viable on a commercial scale of operation.

5.1.3 Clarification

The clarification step C further separates the filtrate stream by removing any remaining large particulate plant material of low value.

This may involve further fine filtering of the filtrate. Alternatively, the clarification step may involve recycling the filtrate for a period of time through the pre-concentration step previously discussed.

5.1.4 De-Watering

After the clarification step, the filtrate is predominately a liquid containing the remnants of the trichomes and a significant water content. The de-watering step D involves the use of conventional filtration techniques as should be known by the skilled addressee and dehumidification to reduce the moisture content and dry the filtrate and leave behind a substantially solid mass of material.

5.1.5 Extraction

To extract target compounds, e.g., cannabinoids from the dried filtrate, known extraction techniques may be employed by the skilled addressee in an extract step E. In one example, a solvent such as ethanol may be used, in combination with filtration techniques.

This may allow the constituents of the trichomes to become aggregated and more easily separated out. The efficiencies that may be achieved with the present disclosure may also require less ethanol to be used at this stage of the process. The filtrate after the preceding steps may have a greater trichome content than with conventional techniques, and as such is more easily separated out.

5.1.6 Refining

The extract of the previous step may be further processed in a refining step F using conventional distillation and/or crystallisation/chromatography techniques that will be readily understood by the skilled addressee.

1.2. System

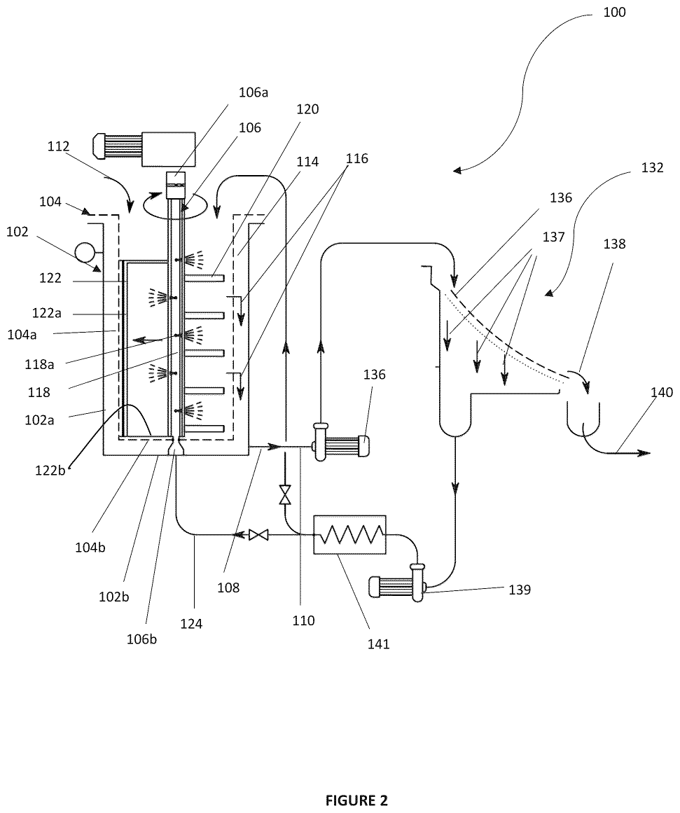

In one example illustrated in FIG. 2, apparatus for use in a system (generally indicated by 100) for processing harvested cannabis in accordance with one aspect of the present technology includes a vessel 102 provided with a filtering medium in the form of a basket 104. In the illustrated example, the system also includes an agitator 106 disposed within the basket. In use, this serves to stir and vigorously agitate the mixture of cannabis plant material and cooling agent when introduced to the vessel, as will be discussed below. However, other examples of the system may employ other means of agitating the mixture; for example, a static mixer (not shown) may be employed downstream of the vessel. Alternatively, a pump (not shown) may be used for this purpose. Furthermore, in some examples, the basket may be omitted and instead a filtering screen may be provided at or proximate the outlet 108 of the vessel to prevent larger particulate cannabis plant material (which may be stalks or the like and thus forming biomass that may be of low value) from exiting the vessel.

The agitation is a notable aspect of the disclosure. Aggressive agitation, compared to the more gentle methods used in the related art, releases more trichomes into the cooling agent. This potentially optimises the extraction of the value components of the cannabis plant material.

The vessel, basket and, if present, the agitator, are arranged and configured to best optimise the implementation of the pre-concentration step of the processing method.

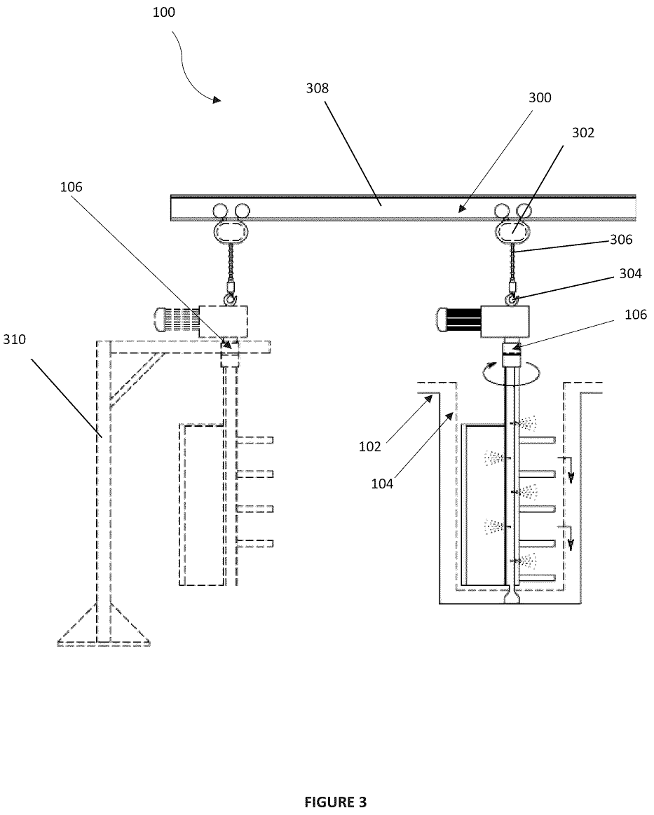

In some examples, the system 100 may also include a transfer mechanism (not shown in FIG. 2 but identified as 300 in FIG. 3) that places and removes the baskets 104 and/or agitators 106 into and out of the vessels 102 as required.

The respective essential and optional parts of the apparatus and system used to perform the processing method shall now be described in detail.

1.2.1. Vessel

Each vessel 102 should be understood to be a container that in use receives the plant material being processed and a quantity of cooling agent. In this example, the separation of the plant material into filtrate, a slurry of plant material and cooling agent, and waste streams takes place within the vessel. However, in some examples, the separation of the filtrate may take place downstream of the vessel.

In one example of the present technology, the vessel 102 is an open tank, with at least its internal surface configured as a cylinder and thus having substantially a circular cross-section. However, this is not meant to be limiting and other shapes and profiles for the vessel will be readily envisaged by persons skilled in the art. The vessel shall now be referred to as a tank throughout the remainder of the present specification.

The tank 102 has a continuous side wall 102a and a base 102b and is fabricated from an appropriate material, such as stainless steel or the like. In use, the tank receives the basket 104; the tank may already be pre-filled with the cooling agent prior to the introduction of the basket or alternatively, the cooling agent may be introduced afterwards.

In an example, the tank 102 has a diameter of approximately 2 metres and has a height that is at least twice its diameter. In this configuration the tank has a volume of approximately at least 4,000 litres. However, it will be appreciated by persons skilled in the art that the dimensions of the tank may be scaled up or down depending on the intended volume of harvested cannabis plant material that is intended to be processed at any one time.

The tank 102 is provided with an outlet 108 as seen in FIG. 2, which in one example is located or otherwise positioned at or proximate the base 102b. This allows liquids received by the tank to be drained away for further processing. It will be appreciated that at least some of the drained liquid is the filtrate, containing the trichomes and their cannabinoids of interest as suspended particulate matter within the filtrate.

The base 102b of the tank 102 may be flat, with the outlet 108 positioned centrally or alternatively as shown in FIG. 2, at a lower edge of the side wall 102a. In other examples, the base of the tank may be in an inverted conical form, with the outlet positioned at the lower most point; this configuration may help with efficient drainage of the liquid contents of the tank.

The outlet 108 is communicative with a discharge pipe 110. This may be under pressure to better optimise flow of the drained liquid and filtrate through the pipes. In some examples, the outlet may include a screen to capture any larger particulate matter that may have escaped the filtering medium and prevent it entering the filtrate.

In some examples, the tank 102 is configured with a supply line or charger (not shown) for the introduction of the cooling agent. This may be to the sides of the tank or alternatively, a separate structure entering the open top of the tank.

1.2.2. Filtering Medium

The filtering medium should be understood to be a container that is configured to act as a filter. In one example, the filtering medium is a basket 104 and shall be referred to as such throughout the remainder of the present specification.

In use, the harvested and pre-processed cannabis plant material is placed 112 within the basket 104. This may be by hand or alternatively may be through the use of a charger or hopper or the like (not shown) as will be readily apparent to a skilled addressee.

It will be appreciated that in some examples of the present disclosure, the basket 104 is configured to be substantially complementary to the tank 102 of the system 100. In a particular example of the present technology, the basket is a cylindrical structure having a continuous side wall 104a and a base 104b. It will be appreciated that in this example, the tank that receives the basket is similarly a cylindrical structure.

In one example, the dimensions of the basket 104 are such that it is 5% to 20% smaller than the tank 102 in which it is to be placed. This means that when centrally located within the tank, there is clearance 114 between the continuous wall 104a and base 104b of the basket and that of the tank.

In one example, the basket 104 may be between 1.9 to 1.4 metres in diameter and 3.8 to 3.2 metres in height. However, it will be appreciated by persons skilled in the art that, as previously noted in respect of the tank 102, the dimensions of the basket may be scaled up or down depending on the intended volume of cannabis plant material that is intended to be processed at any one time.

In some examples, the base 104b of the basket 104 may be configured with structures that are complementary to structures provided to the upper surface of the base 102b of the tank 102. This helps to anchor the basket relative to the tank, particularly if the contents of the basket are being stirred through the action of the agitator 106.

In one example of the present technology, the basket 104 is fabricated from stainless steel sheet having perforations of approximately 0.3 mm to 3 mm. In other examples, the walls 104a and base 104b of the basket are fabricated from stainless steel mesh.

Once filled with the plant material to be processed, the basket 104 is transferred to the tank 102 for the pre-concentration phase. Alternatively, the basket is placed within the tank and freshly harvested and pre-processed cannabis plant material is then introduced to the basket. The tank may already contain the cooling agent prior to the basket being placed therein or alternatively the cooling agent is introduced to the tank after the basket.

It will be appreciated that, in use and following the introduction of the cooling agent, the basket 104 serves as a filter for the plant material being processed. The larger particulate matter forms the waste material that remains within the basket while smaller particulate matter and dissolved compounds, including the parts of particular value, the trichomes, forms the filtrate 116 which passes through the walls 104a of the basket and is allowed to exit the tank 102 via the outlet 108 along with the cooling agent. The filtrate is largely a slurry of shredded plant material, broken up into particulate matter through the action of the agitator, and cooling agent.

In other examples not shown here, the filtering medium may be a screen or the like, positioned at or proximate the outlet of the tank. The screen is dimensioned to exclude larger particulate plant matter, which is likely to be low value biomass, from exiting the tank, but smaller particulate matter and dissolved compounds, such as trichomes, may pass through along with the cooling agent.

1.2.3. Agitator

In one example of the present technology and as shown in FIG. 2, the system 100 includes an agitator 106. In use, the agitator is configured to agitate and mix the cooling agent and harvested cannabis plant material in the filtering medium 104.

As discussed above, the tank 102 may be filled with a cooling agent. In this example, the action of the agitator serves to provide the necessary forces to encourage physical breakdown of the plant material. In particular, the agitator encourages separation of the now significantly cooled trichomes from the remaining plant material so that they become entrained in the cooling agent as a suspended particulate matter. This is a notable aspect of the present technology.

In the illustrated example, the agitator 106 comprises an elongate, vertically orientated shaft 118. A plurality of agitating blades or tines 120 extends substantially perpendicularly from the shaft at regular intervals.

The agitator 106 is provided with a drive motor 106a to rotate the agitator about a vertical axis. This action, with the blades 120, serves to mix and agitate the immersed plant material and encourage the separation of same into the low value waste and the high value filtrate.

In examples, the motor 106a acts to rotate the agitator 106 at speeds ranging from 20 to 200 revolutions per minute or more. The higher speeds are optional for aggressive agitation and breakdown/shredding of the immersed plant material to release the trichomes containing the cannabinoids of interest. It can also help in breaking down of the trichomes into fragments. A downstream effect of this is that less time is required in further processing to separate out the trichomes and fragments of same from the rest of the plant material.

In some further examples, the motor 106a may be set to provide, at regular intervals, a pulsing action where the speed of rotation is increased for a period of time. Alternatively, the motor may be operative such that for a period of time, the agitator rotates in a first direction, and then a second direction. In both of these examples, this enhances the effect of the agitator upon the plant matter.

In some further examples not shown here, to supplement the effect of the agitator, the blades may be configured with tines or the like extending in various orientations to further thresh the plant material in various directions when the agitator is in motion.

In the illustrated example, the base 106b of the agitator 106 may be configured to key into or otherwise engage with the basket 104, optionally at the base 104b of the basket. In some examples, this may be by way of a bearing hub or the like. This helps ensure the structural integrity and minimise stresses during operation of the agitator.

In one example, at least a portion of the shaft 118 of the agitator 106 is hollow, with a series of apertures 118a allowing communication between the interior of the shaft to the exterior, arranged at regular intervals along its length. In this example, the agitator is provided with a pipe 124 for pressurised chilled water which is directed through the hollow tube of the shaft and out the apertures provided along same. In some examples, the apertures may be configured with nozzles or similar structures to help optimise the desired radial flow path.

In use, the pressurised water that is directed through the shaft 118 of the agitator 106 and emitted out the apertures 118a serves two purposes. Firstly, depending on the extent of the pressure at which it exits the shaft of the agitator, it provides some impact forces on the plant material, supplementing the action of the agitator and assisting in the separation of trichomes from the plant material. Secondly, it creates a radial flow path within the basket and tank, encouraging further separation of the finer particulate plant matter, including the trichomes, from the plant material being processed and directing this towards the space 114 between the basket 104 and the inner surfaces of the tank 102.

In some examples, the cooling agent may be introduced to the tank via the hollow shaft 118 of the agitator 106 as shown in FIG. 2 but in other examples, as previously discussed, the cooling agent may be delivered to the tank via a separate charger or supply line.

In a further example, the agitator 106 may include a scraper 122 which, in use, bears against the continuous wall 104a of the basket 104 and scrapes its surface. In one form, the scraper is an elongate bar 122a mounted to a pair of arms 122b extending perpendicularly from the shaft of the agitator; as the agitator rotates, so does the scraper. This helps optimise the filtering of the plant material performed by the continuous wall and/or base of the basket, minimising any blockages that may occur.

In some examples, the scraper 122 may be configured to receive a plastic insert (not visible) which is the scarping surface that contacts the continuous wall 104a of the basket 104 in use. In this form, the use of the plastic insert reduces the risk of damage to the walls of the basket. In some examples, the scarper 122 may be arranged to encourage the development of the radial flow path that directs the filtrate 116 towards the space 114 between the basket 104 and tank 102.

1.2.4. Transfer Mechanism for Basket and/or Agitator

In one example of the present technology as shown in FIG. 3, the system 100 is provided with a transfer mechanism 300 to facilitate the placement of the basket 104 and/or agitator 106 within the tank 102. On the left in dashed lines is an agitator in a stored position and on the right an agitator shown in an “in use” position, where it is disposed within the tank.

In its simplest form, the transfer mechanism 300 includes lifting equipment in the form of a winch 302 provided with a hook 304 and cable 306 which engage with complementary fittings (not visible) provided to the upper surfaces of the agitator 106 and baskets 104. In alternative examples not shown, the cable may be provided with a loop which engages with hooks or similar fittings provided to the agitator and baskets. The skilled addressee should readily envisage and implement a transfer mechanism suitable for use with the present system.

In some examples, the transfer mechanism may include a gantry 308 along which the lifting equipment 302 is able move. This allows the basket 104 and/or agitator 106 to be moved to a location clear of the tank 102.

In some examples, the agitator 106 may be provided with a frame or support stand 310 for when it is not in use. When the agitator is located within the frame, the transfer mechanism 300 may then be used to place the basket 104 in the tank 102 and/or remove it from same.

1.2.5. Separator

In one example of the present technology as shown in FIG. 2, the system 100 includes a separator 132 which will be understood to be downstream of the tank 102 and receives laden quantities of filtrate 116 via the outlet 108 and discharge pipe 110 of the tank. The separator will be understood to be an apparatus that processes the filtrate into a high value stream and a low value stream; the former includes the trichomes of interest while the latter may be spent or partially spent cannabis plant material from which at least some of the trichomes have been removed.

The discharge pipe 110, pressurised by a pump 134, deposits the filtrate to and through a filter screen 136. In this example, the screen includes apertures ranging in size from one to 200 microns. The filter screen therefore captures relatively large components in the filtrate. Surplus liquid, i.e. cooling agent, passes through the filtering surface and is pumped back to the tank. In conventional systems, this surplus liquid is regarded as a waste stream and is discharged. In the present disclosure it is recognised that there may be still value components entrained in the surplus liquid, and hence returning it to the tank provides a further opportunity for extraction of those value components.

The filtrate may still contain target compounds such as cannabinoids. These compounds can be subsequently removed from the filtrate by additional processing steps (as are discussed in more detail below or as should be known to the skilled addressee).

The use of the separator 132 further processes the filtrate by separating out at least some of the target compounds from the filtrate, e.g., the trichomes or relatively large aggregates. It also serves to filter out at least some of the cooling agent 137 used in the method. This cooling agent passes through the filter screen and may be collected and recycled into one or more of the tanks 102 for re-use for further processing as required. As noted previously, this is an advantage of the disclosure, since it provides further opportunity to capture any value components, trichomes or fragments of trichomes, missed by the separator, minimises wastage of the cooling agent, and also optimises the processing of the plant material. As shown in FIG. 2, this is via the pipe 124 that is communicative with the shaft 118 of the agitator 106 (or it could just as easily be via a separate pipeline back to the tank 102). In doing so, the cooling agent is pumped 139 through a chiller 141 to reduce its temperature.

The remaining filtrate 138, once processed by the separator, flows off the screen 136 and is collected for further processing (140—for example, the clarification and/or dehumidification steps previously referred to). Instead of an angled filter screen, as shown in FIG. 2, in one form the separator may be one or more cyclonic separators. In other forms, the separator may comprise one or more rotary or fixed filters, with a screen of between 1 to 200 microns. Persons skilled in the art will readily utilise and/or adapt known separators suitable for use in the present system.

1.2.6. Recirculation Loops/Pathways

Not all of the trichomes, especially if broken up into their constituent parts, are captured when the filtrate is passed over the separator. To optimise the extraction of these value components, the trichomes containing the desired CBD and THC, from the plant material being processed, the system as previously described may be provided with one or more recirculation loops to direct what may otherwise be deemed as a waste stream from the separator for further processing by the present disclosure. The system may also be provided with additional means of agitating the filtrate following its exit from the tank prior to being introduced to the separator.

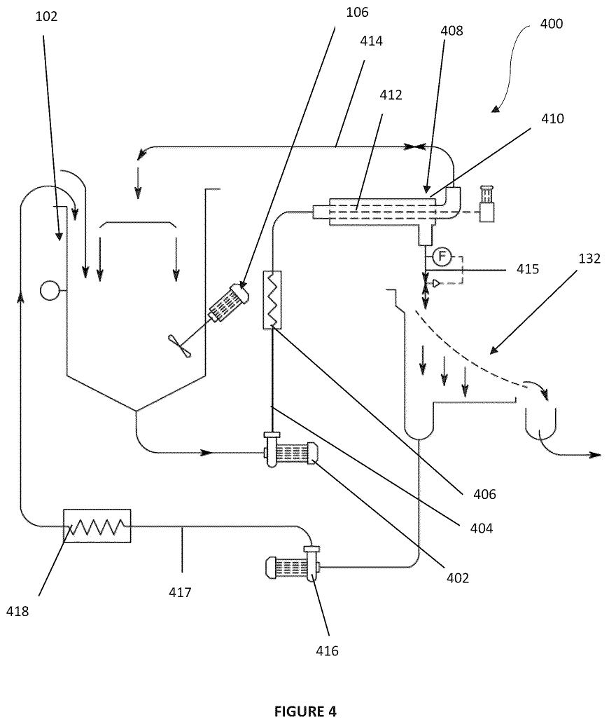

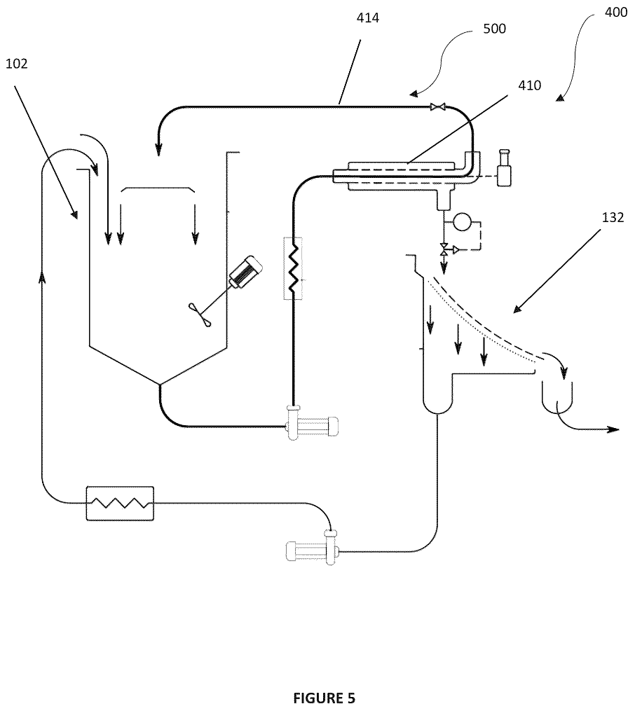

An embodiment of the disclosure employing such recirculation loops is described in FIGS. 4 to 7. Referring firstly to FIG. 4, this shows a schematic of a system 400 (features common to the embodiment of the system previously described in FIG. 2 use like numbers) with recirculation loops 500 and 600 and pathway 700. These are shown in bold in FIGS. 5, 6 and 7 respectively.

The system 400 includes a tank 102, which in use contains a slurry of the cooling agent and harvested cannabis plant material (not shown). Once the tank has been filled to the prescribed level, the agitation of the slurry through the action of an agitator 106 may commence after a period of time has elapsed. This is to ensure that the slurry has cooled sufficiently for the trichomes to become brittle and potentially more easily separated from the rest of the plant material and/or broken up and fragmented.

In operation, the agitator 106 acts upon the slurry as previously described, breaking up the plant material, separating the trichomes from the leaves, and potentially assisting in the breakdown of the trichomes into fragments or even their constituent components, which occurs when the trichomes break open and release the cannabinoids, in oil form, into the cooling agent. This action is performed for a designated period of time, optionally for at least 30 to 90 minutes. It should be appreciated that aggressive agitation of the slurry can potentially reduce processing time compared to conventional systems.

The larger particulate plant material is retained within the tank 102 by a filtering medium (not shown) while the filtrate, a liquid mixture of cooling agent, with entrained smaller particulate plant matter, including the trichomes and their fragments, not captured by the filtering medium, exits the tank and through the use of a pump 402 is passed along a pipeline 404 through to a static mixer 406. The action of the pump upon the filtrate helps with the separation and breakup of the trichomes within the filtrate. The action of the static mixer has a similar effect upon the filtrate. This repeated aggressive agitation, firstly in the tank through the action of the agitator and during the subsequent transfer of the filtrate from the tank to the separator is notable and helps optimise efficiencies.

In some examples, not shown here, the agitator 106 may not be present in the system 400. Instead, the agitation of the filtrate may be performed through the action of the pump 402 and/or the static mixer 406. In other examples, an agitator, pump and static mixer may all be present in the system; this may mean a particularly aggressive agitation of the filtrate can be achieved, further optimising processing time.

From the static mixer 406, the filtrate is passed through a screen 408 with apertures ranging in size from one to 200 microns (50 to 150 microns is optional) in order to separate at least a portion of the liquid content, which is essentially the cooling agent, i.e. chilled water, from the particulate plant matter entrained within, much of which is the valuable trichomes.

In the illustrated system 400, the screen 408 is a scrape surface filter 410. This should be understood a screen 412 with a mechanical scraper (not visible) acting against the screen to minimise or prevent build up of solid matter. It should be noted that this is not intended to be limiting and other screening apparatus may be used.

In this manner, the majority of the filtrate that was transferred from the tank 102, at least 20% to 70%, which is largely cooling agent, is separated and returned to the tank; the remaining 30% to 80% of the filtrate, with a much high percentage of solid matter relative to liquid, is transferred to the separator 132.

The return of the majority of the filtrate in this first recirculation loop 500 is achieved through the use of a pipeline 414 extending between the scrape surface filter 410 and the tank 102. The pathway of the first recirculation loop, from tank to the scrape surface filter and back to the tank is shown in bolded lines in FIG. 5. It should be appreciated that there may be considerable inefficiency in the filtering action of the scrape surface filter 410. The use of the first recirculation loop allows the greater portion of the filtrate to be returned to the tank for further agitation and breakup of the entrained plant matter therein. Additionally, some trichomes will have been fractured or broken open and fragments of same may still be entrained, having failed to be captured by the scrape surface filter 410. In some examples, these may be extracted from the cooling agent by further processes before the filtrate re-enters the tank 102. The extracted trichomes and/or cannabinoids may then be added to those extracted out through the action of the separator 132.

In some examples, concurrently with this first recirculation loop 500, more freshly harvested cannabis plant material may be introduced to the tank 102 via a charger (not shown) or a worker (not shown) depositing it into the tank. This allows the system 400 to operate nearly continuously, until there is an excessive amount of large particulate matter within the tank. This may need to be cleared so that the tank can be cleaned and prepared for a fresh batch of harvested cannabis plant material.

As noted above, the remaining filtrate is passed through to the separator 132. The flow rate of the filtrate from the screen 408 to the separator may be controlled through valve 415 to help ensure that there is no overload, which could otherwise compromise the efficiency of operation. Still having a large liquid content, the separator acts to further reduce the amount of water present in the filtrate.

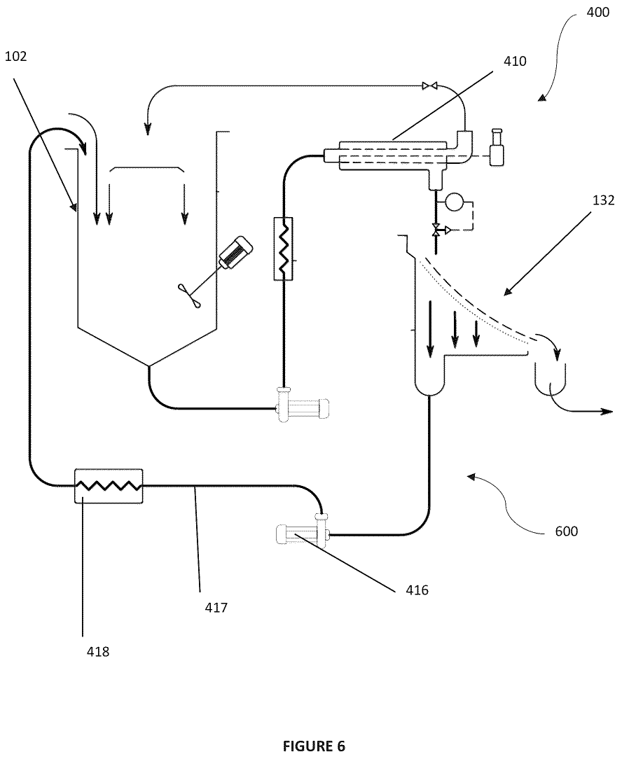

The water extracted at this stage is fed back into the tank 102 via a second recirculation loop 600, as shown in bolded lines in FIG. 6. Of the portion of the filtrate that reached the separator 132, around 30% to 80%, through the use of pump 416 is returned to the tank via a pipeline 417. This portion of the filtrate is largely made up of cooling agent although some trichomes will have been fractured or broken open and fragments of same may still be entrained, having failed to be captured either by the scrape surface filter 410 or the separator.

In some examples, as with the recirculation loop described in FIG. 5, the entrained fragments of trichomes and/or the contents of the trichomes may be extracted from the cooling agent by further processes before it enters the tank 102.

Before its reintroduction to the tank 102, this portion of the filtrate is passed through a chiller 418 to bring its temperature back to 0° Celsius, or as close as possible to 0° Celsius.

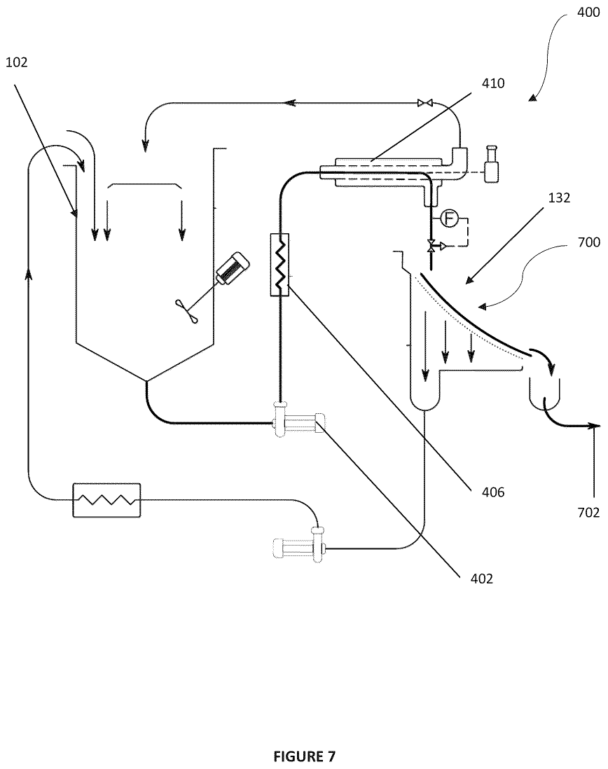

In FIG. 7, a portion is of the filtrate is separated out through the action of the separator 132 of the system 400. This filtrate is largely a solid resinous mass 702 of trichomes and/or cannabinoids. This final portion of the filtrate may form as little as 5% to 20% of the original amount transferred from the tank 102, the balance being the liquid portion that has been returned to the tank through the use of the first 500 and second 600 recirculation loops. The pathway 700 of the final portion of the filtrate from the tank 102, pump 402, static mixer 406 and scrape surface filter 410 to the separator is shown in bold. This mass is collected and then transferred for further processing, for example, de-watering and extraction of the CBD and THC with solvents.

It should be appreciated that in the system 400 of FIGS. 4 to 7, the first and second recirculation loops 500, 600 are operated concurrently with pathway 700. This optimises processing of the harvested cannabis plant material while minimising wastage of both cooling agent and plant material.

1.2.7. Operation

The operation of an individual tank of the present disclosure as illustrated in FIG. 2 involves certain key steps. For sake of understanding the operation, and only by way of example, the explanation assumes the tank is dimensioned to have an internal diameter of approximately 2 metres and an internal height of approximately 4 metres, giving an overall operating volume of approximately 4,000 litres. As previously noted, the skilled addressee will readily understand that the tank may be scaled up or down depending on the volume to be processed and therefore may adjust the timing of the operation accordingly.

1.2.7.1. First Step

To begin the process, a batch of the cannabis plant material to be separated out into waste and filtrate is introduced into the tank 102, separately or concurrently with an appropriate amount of cooling agent. The batch is allowed to be fully immersed in the cooling agent, a slurry of chilled water and ice cubes in this example, and have its overall temperature reduced to the point where the trichomes become brittle and more easily separated from the remaining plant material.

In one example, depending on rate of charging of the plant material and chilled water, this first step may take up to 30 minutes.

1.2.7.2. Second Step

The mixture of plant material and chilled water and ice is then vigorously agitated through the use of the agitator 106 and allowed to circulate within the tank 102 and basket 104. This action helps to separate the smaller particulate matter, including the trichomes, from the larger portions of the plant material being processed and into a filtrate.

Throughout this process, the filtrate is allowed to exit the tank 102 via the outlet 108 and be directed to the separator 132. At the separator, the filtrate is further filtered using the filter screen 136 to extract at least a portion of the trichomes from same. These trichomes may be entrained in at least some of the chilled water used in the process.

The remainder of the filtrate, which may comprise cooling agent, particulate plant material, suspended solids (including any trichomes not extracted from the filtrate by separator 132) is directed or otherwise recirculated, via pump 139 or gravity feed, back to the tank 102. In conventional systems, this may be considered waste but it has been found that there can still be a significant amount of trichomes present in this filtrate, or able to extracted from the filtrate, to justify its further processing by returning to the tank.

In one example, the mixture is agitated and recirculated for approximately 90 minutes.

1.2.7.3. Third Step

Once the mixture has been agitated and recirculated for the appropriate period of time, the tank 102 can be emptied. This involves the draining of the liquid portion of the mixture, containing the filtrate, through the outlet 108 and to the separator 132.

The residual plant material, the waste from the pre-concentration step, is left behind in the basket 104 which is transferred out of the tank 102. This allows the waste to be removed.

In one example, this step may take up 11 minutes.

1.2.7.4. Fourth Step

To process another batch of harvested cannabis plant material, it is necessary to rinse or otherwise clean the basket and tank with water, together with, or instead of, a cleaning agent such as a mild detergent or solvent. This water is delivered through the agitator 106 (or separate supply if present) and allowed to drain through the outlet 108 of the tank 102, be removed from the system, and then recirculated as a rinsing step as desired.

In one example, the cleaning step may take up to 19 minutes, before the tank 102 is ready to process another batch of harvested cannabis plant material.

In the recited example of the operation of the system, it takes approximately 150 minutes to cycle a single batch of freshly harvested cannabis plant material and the required cooling agent (collectively, approximately a volume of 4,000 litres). Over a 24-hour period, this allows for an hourly processing rate of approximately 1,600 litres through the tank.

1.3. Processing Modules

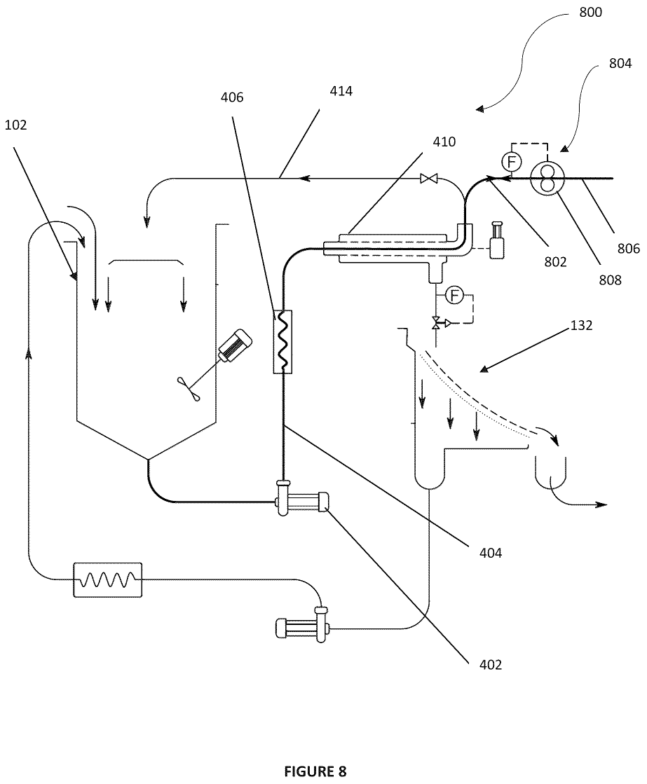

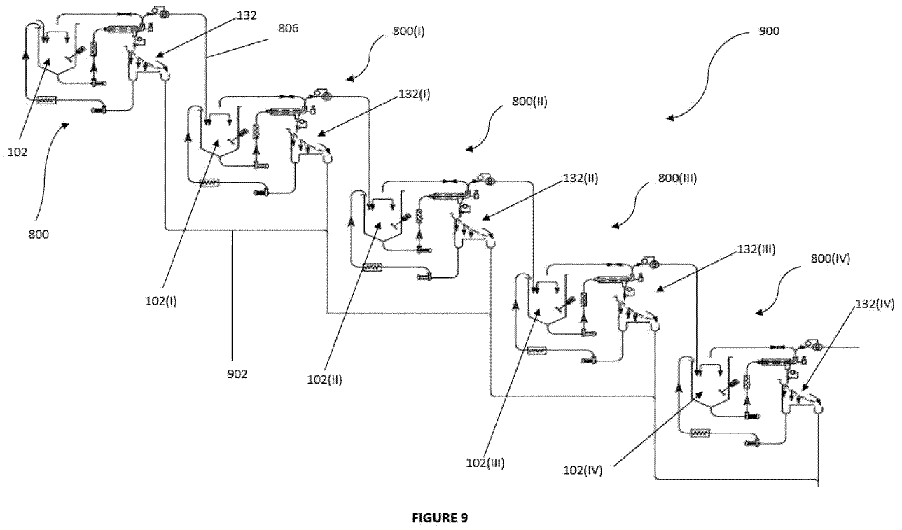

As will become apparent from the discussion of FIGS. 8 and 9, in a further example of the present technology, the apparatus and system described in FIGS. 4 to 7 may be employed as a discrete processing module 800 within a continuous processing system 900.

In this example and as shown in FIG. 8, the processing module 800, is largely as previously described in FIGS. 4 to 7 (like numbers are used for features common to both examples) and includes a tank 102, agitator 806, pump 402, static mixer 406, and scrape surface filter 410. It also has the various recirculation loops and pathways described in FIGS. 5 to 7, allowing for at least a portion of the filtrate to be returned to the tank for further processing or passed to the separator 132 to extract the value components.

In addition, an outlet 802 for a further pathway 804 is provided on the pipeline 414 leading away from the scrape surface filter 410 and returning to the tank 102. This outlet is part of a conduit 806 linking the processing module 800 of FIG. 8 to an adjacent processing module (not shown).

This outlet 802 allows at least a portion of the filtrate passing through the pipeline 414 to be removed as an outflow, via a positive displacement pump or the like 808 and transferred via the conduit 806 as an inflow to the adjacent processing module, which has all of the features, tank, agitator, separator and so on, of the processing module 800 of FIG. 8. The remainder of the filtrate passing through the pipeline may be returned to the tank 102, as described in respect of FIG. 5.

However, it should be appreciated that alternatively, all of the filtrate passing through the pipeline 414 could be removed through the outlet and transferred to the adjacent processing module rather than just a portion of the filtrate. This may be achieved through the use of a valve (not shown) or such like positioned on the pipeline.

FIG. 8 shows the outlet 802 as being located on pipeline 414. This means that the filtrate that is removed and transferred to the adjacent processing module has been substantially mixed and already partially filtered by virtue of passing through and being processed by the pump 402, static mixer 406 and scrape surface filter 410. It is a more refined and concentrated filtrate at this stage compared to what was originally allowed to exit the tank 102. As the transferred filtrate is processed by the adjacent processing module, the extraction of the value components is likely to be more efficient.

The outlet 802 is optionally located on the pipeline 414 leading back to the tank 102 for maximum efficiency. However, it should be noted that it may alternatively be located on pipeline 404, for example after the static mixer 406 but before the scrape surface filter 410 to remove a portion of the filtrate before it reaches the scrape surface filter. This will mean that the portion of filtrate that is passed to the adjacent processing module may not be as refined.

In this example, the continuous processing system may use a plurality of processing modules 800, 800(I), 800(II), 800(III), 800(IV) in series as shown in FIG. 9.

Prior to it being passed to the separator 132, at least a portion of the filtrate that is processed by the first processing module 800 is transferred to the second processing module 800(I) via conduit 806. This is introduced directly to the tank 102(I) of the second processing module. Similarly, at least a portion of the filtrate processed by the second processing module is transferred to a third processing module 800(II) and so on.

It will be understood that the continuous processing system 900 is operated such that the second 80(I) and successive processing modules receives the outflow from the previous processing modules, i.e. the tank 102(I) in each processing module is being continuously charged. This helps optimise the processing of the plant material by ensuring as much trichomes as possible to extracted while still being efficient with water usage.

This method of operating the system 900 of FIG. 9 means that the portion of filtrate that is passed from processing module to processing module becomes more and more refined and filtered, containing increasing amounts of value components relative to the cooling agent. Thus, increasing efficiencies of separation of the value components from the filtrate may be achieved at each successive processing module. This maximises the extraction of the value components. This efficiency may also have a bearing on the operating parameters of the various processing modules. For example, the processing times at each successive processing module may be reduced; for example, the agitation step at the third processing module 800(11) may not need to be as prolonged as was used at the first processing module 800. Similarly the separator 132(11) may not need to be operated for as long as the separator 132 of the first processing module.

A further consideration is that if the proportion of the filtrate that is removed and transferred to the next processing module remains the same, this will mean that the volume of the filtrate processed by each processing module may decrease. This may have a bearing on the size of successive processing modules as they may be decreased in capacity accordingly. Conversely, the operator may choose to gradually increase the proportion of the filtrate removed at successive processing modules such that the volume of filtrate being processed remains substantially constant.

Alternatively, in some instances, the operator of the system 900 may choose to supplement the filtrate as it is introduced to each successive processing module. It will be appreciated that without being supplemented, the quantity of filtrate may be significantly reduced in volume by the time it has reaching the fifth processing module 800(IV) and so additional cooling agent may be introduced to the tank 102(IV) as the filtrate is added. This will have the effect of diluting the filtrate and thus may require an adjustment in the operating parameters; for example, the agitation of the filtrate may need to be prolonged.

The extracted value components retrieved by the separator 132 of each processing modules 800 to 800(IV) is drained to a common line 902 and transferred for further processing and treatment, for example, the de-watering step discussed in respect of FIG. 1.

In some examples of the present technology, the rate of outflow (the filtrate being removed by the separator and the residual cooling agent/harvested cannabis plant material) from each processing module 800 to 800(IV) of the processing system 900 is substantially consistent with the rate of inflow (the residual cooling agent/harvested cannabis plant material from the previous processing module) into that module.

As such, each processing module handles a similar volume of material at any one time such that it can be operated at its maximum capacity at all times for efficiency. It will be appreciated that this may affect the amount time the mixture of cooling agent/harvested cannabis plant material is agitated.

However, in some alternative examples, where the outflow from the processing modules may be greater than the inflow, each successive processing module after the first may be provided with means to allow supplementary cooling agent and/or harvested cannabis plant material to be added to its respective vessel to ensure it is operating at maximum capacity. As filtrate is removed from each module, the volume remains substantially unchanged since it is replaced with cooling agent and/or harvested cannabis plant material as required.

In some examples, on initial start-up of the continuous processing system 900, only the first processing module 800 is charged with cooling agent and harvested cannabis plant material. Once it has reached its operational capacity, the outflow from the first processing module serves to charge the second processing module 800(I); once it has reached operational capacity, the outflow from the second processing module serves to charge the third processing module 800(11), and so on. This means, depending on the rate of outflow, it may be several minutes or hours before all the modules of the continuous processing system are actively processing the filtrate and extracting the value components.

For example, each vessel of the five processing modules 800 to 800(IV) of the continuous processing system 900 of FIG. 9 has an operational capacity of 1000 litres. The inflow of cooling agent and harvested cannabis plant material may be provided to the upstream processing module, i.e. the first processing module 800, at a rate of 5000 litres per hour; at this rate, the vessel of the first processing module reaches operational capacity in 12 minutes, at which time the outflow to the vessel of the second processing module 800(I) commences. This too reaches operational capacity after 12 minutes and so on. This provides a residence time for the inflow of cooling agent and harvested cannabis plant material in each processing module of 12 minutes and a total process time of 60 minutes at the final downstream processing module, i.e. the fifth processing module.

Of course, simultaneously with the outflow, a portion of the contents of each vessel is diverted to the separator of each processing module 800 to 800(IV). This may be set at a proportion of the inflow; for example, at 0.5% of the inflow rate, 25 litres an hour may be diverted to the separator 132, the balance being transferred to the following processing modules 800(I) to 800(IV). If this is consistent for each of the five processing modules, this leaves the final outflow from the last processing module as 4875 litres per hour. This outflow would be transferred for further separation.

Optionally, the volume of outflow diverted to the separator increases for at least one or more successive processing modules 800(I) to 800(IV). This may be achieved by increasing the proportion drawn off from the vessel of the processing module, for example from 0.5% at the first processing module 800 to 5% of the initial inflow rate at the third processing module 800(III), or by increasing the overall volume of the mixture of cooling agent and harvested cannabis plant material at the processing module 800(111) by introducing additional cooling agent.

The rate at which this occurs may vary depending on the relative position of each processing module 800 to 800(IV) within the overall continuous processing system 900. For example, in the first processing module 800, the discharge from the vessel 102 to the separator 132 may be at a rate of 25 litres an hour, when set at 0.5% of the initial inflow rate. This means that the outflow to the second processing module 800(I) is 4975 litres an hour. The second processing module is similarly set such that the outflow to the third processing module 800(II) is 4950 litres an hour. However, at the third processing module, the discharge from the vessel 102(II) to the separator 132(11) may be set at 5% of the initial inflow rate; this means that 250 litres an hour is being diverted to the separator of the third processing module while 4700 litres an hour is transferred to the fourth processing module 800(111). Alternatively, the discharge from the vessel to the separator may remain at 0.5% of the initial flow rate but a charge of 250 litres of cooling agent is added such that 275 litres an hour is removed to the separator. The fourth and fifth processing modules 800(IV) may be similarly set such that the final outflow from the last processing module is 4125 litres an hour, with 875 litres an hour in total being extracted to the separators of each processing module.

The agitation may be more severe at the later processing modules relative to the earlier processing modules, as the cannabis plant material becomes more and more broken down. This reflects the relative efficiency of the extraction and breakdown of the trichomes at each successive processing module.

In other examples, in addition to the first processing module 800, some or all of the processing modules 800 to 800(IV) of the continuous processing system 900 may be charged with cooling agent and harvested cannabis plant material on initial start up. In these examples, the value components may begin to be extracted from the filtrate sooner although the increasing amount of concentrated filtrate being passed as inflow into successive processing modules takes longer to be achieve.

The skilled addressee will appreciate that the recited examples of the continuous processing system 900 are not meant to be limiting and variances from what is described are envisaged, depending on available space constraints and desired operating times. For example, in some embodiments, the vessels in the later downstream processing modules may dimensioned smaller than the earlier upstream processing modules. This may be optional at high filtration rates, where the proportion of the volume of cooling agent and harvested cannabis plant material removed at each processing module is relatively high such that the downstream processing modules are not required to have such a large operational capacity. In other examples, the vessels of the downstream processing modules may be larger, to facilitate increased residence times.

5.4 Other Examples of the Technology

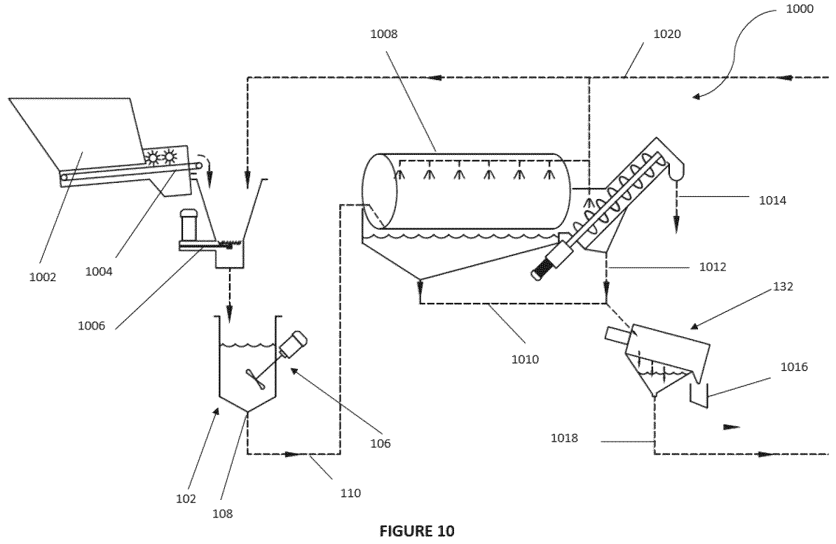

An alternative embodiment of a system for processing harvested cannabis is shown as a schematic in FIG. 10 (like numbers are used for features common to previous embodiments described). In this embodiment, the system 1000 includes a biomass feeder 1002 in which the harvested cannabis plant material (not shown) to be processed is deposited.

The harvested cannabis plant material (not shown) deposited into the biomass feeder 1002 may include significant amounts of stalks and fan leaves which ideally should be removed or at the very least significantly reduced to increase the relative proportion of the plant material, the buds, flowers and sugar leaves, these containing the trichomes, undergoing the pre-concentration step (as discussed in respect of step B in FIG. 1).

The harvested cannabis plant material is delivered via a conveyor 1004 to a mill 1006. This breaks the plant material down to much smaller sizes, optionally to around 3 to 5 mm. The milled harvested cannabis plant material (not shown) is then passed to a vessel/tank 102 containing cooling agent in the form of chilled or iced water, or to which cooling agent is subsequently added. In some embodiments not shown here, the stalks and fan leaves may be removed prior to the cannabis plant material being deposited into the biomass feeder. This may optimise the operation of the mill.

The agitator 106 further breaks up the plant material and forms of a mixture of harvested cannabis plant material and cooling agent. The mixture contains a relatively large amount of the cannabis plant material, and be a relatively thick fluid, e.g., a slurry. The aggressive agitation of the harvested cannabis plant material to break it up in shreds and particulate matter helps with separating out the trichomes in a more time efficient matter.

The mixture is allowed to exit the tank via an outlet 108 that is communicative with a discharge pipe 110 that leads to the first of two separators 1008, 132. The use of at least two separators helps to further optimise the extraction of the trichomes from the harvested cannabis plant material being processed.

Prior to being introduced to the first separator 1008, the mixture may be passed through a large filter screen (not shown in FIG. 10) to exclude the largest of the particulate cannabis plant material entrained therein. This screen may be at or proximate the outlet 108. Alternatively, as with the embodiment described in respect of FIG. 2, a filtering container or basket may be present (not shown in FIG. 10), the agitation of the harvested cannabis plant material taking place in this. The largest of the particulate cannabis plant material may be retained within the basket. Of course, in some embodiments there may be no screening of the mixture taking place prior to being introduced to the first separator.

Once the system 1000 is operating, the rate at which the mixture is transferred from the vessel 102 to the first separator 1008 is ideally substantially matched by the rate at which cannabis plant material and cooling agent is added to the vessel 102. This allows a continuous feed into the first separator; similarly, there is continuous outflow from the first separator to the second separator 132. This substantially constant throughput is ideal for operating efficiencies. In related systems, throughput is constrained because of the gentle agitation that is used to separate the trichomes from the cannabis plant material. In the present system, the aggressive agitation is such that breaks up the plant material an expeditious manner. This facilitates better matching of the output of the separators can be matched to that of the vessel.

The first separator 1008 separates the mixture into a first stream containing trichomes and cooling agent and a second stream containing harvested cannabis plant material. The first stream has two pathways 1010, 1012, to the second separator 132. The second stream 1014 may be removed from processing as spent cannabis plant material from which the majority, if not all, of the trichomes have been removed. At the second separator, further working of the first stream is performed, to separate it into a first portion 1016, containing trichomes, and a second portion, which is transferred for further processing via pipeline 1018. Much of the second portion, which is substantially a liquid, is cooling agent but this is likely to still contain trichomes and fragments of trichomes that was not captured by the second separator. Further processing of the cooling agent, e.g., filtering via a concentration membrane or similar equipment (not shown) as will be apparent to a skilled addressee may be performed to claim the trichomes contained in the second portion.

The cooling agent may be reclaimed in the further processing for reuse in the processing module 1000. This is advantageous as it reduces potential water wastage. In one example, the reuse of the cooling agent may involve returning it via pipeline 1020 for reuse in the processing module 1000, for example as a cooling agent at the milling machine 1006 or as a rinse agent at the first separator 1008 (as explained in further detail below).

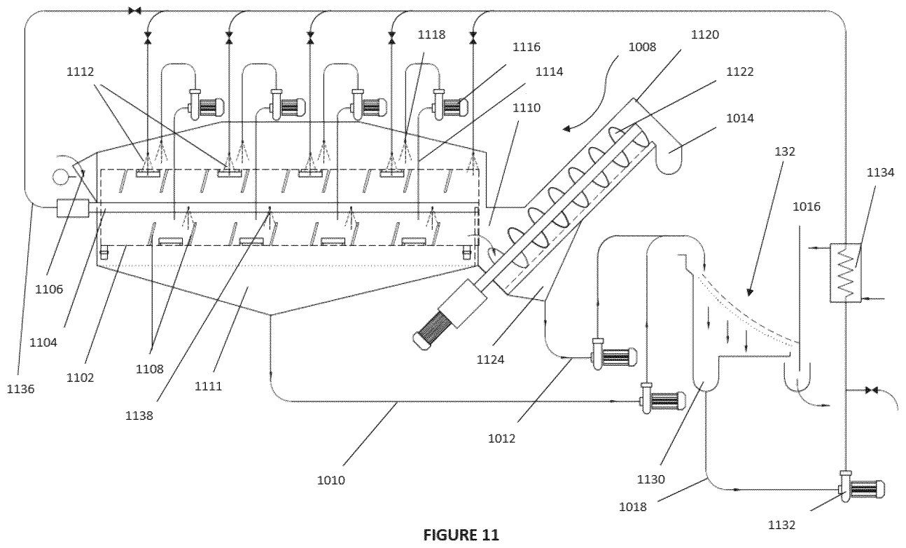

Referring now to FIG. 11, the workings of the two separators 132, 1008 are explained in further detail. In this example, the first separator 1008 is in the form of a rotary screen 1102, which is substantially a cylindrical mesh structure arranged to rotate about a horizontal axle 1104. The mixture of agitated harvested cannabis plant material, having been thoroughly agitated and broken up into fragments and particulate matter in the vessel, is received at the upstream end 1106 of the screen. The rotary screen is provided with tines 1108 at regular intervals which urge the material passing through to the downstream end 1110, stirring and agitating it as it does so. Cooling agent is able to exit the screen and drains to a collection area 1111.

It should be appreciated that the cooling agent in the collection area 1111 may contain some high value components, i.e. trichomes, of fragments of same, entrained therein, or individual cannabinoids or substances naturally produced by the cannabis plant. A pathway 1010 is provided between the collection area to transfer the cooling agent to the second separator 132, which may be in the form of a rotary drum sieve or the like for further processing and capture of the value components.

Depending on its configuration, the lower portion of the rotary screen 1102 may be partially immersed in the cooling agent (chilled or ice water). A rinse agent, in the form of additional cooling agent, is continuously delivered to the screen via wash entry points 1112, i.e. spray heads or nozzles. These serve to rinse down the exposed surfaces of the screen. Additionally, if the lower portion of the rotary screen is sufficient immersed, cooling agent may be withdrawn from this area along a pipe 1114 via a pump 1116 and returned to the screen at outlets 1118 at the upper portion.

At the downstream end of the rotary screen 1110 the separator 1008 is provided with a conveyor 1120. This is arranged to separate the agitated harvested cannabis plant material passing therethrough into a first or high value stream, containing the value components such as trichomes, cannabinoids and other naturally occurring compounds, and a low value stream 1014 containing mostly spent cannabis plant material from which at least a portion of the value components have been separated.

In the illustrated example, the conveyor 1120 includes a helical auger 1122 which has been inclined. As the cannabis plant material is conveyed upwards to the upper end at which the low value stream 1014 emerges, cooling agent containing entrained trichomes and fine particulate matter is able to drain towards the collection area 1124 at the lower end of the conveyor. However, it should be appreciated that other forms are envisaged for the conveyor including, for example, a belt conveyor (not shown) working in combination with press rollers (not shown) or the like acting upon the cannabis plant material on the belt. This extrudes the liquid component, i.e. the cooling agent and entrained value components therein.

From the collection area 1124 of the first separator 1008, the cooling agent and value components is transferred to the second separator 132 via pathway 1012 for further separation into a substantially resinous or solid mass, which contains the value components, i.e. trichomes 1016, and cooling agent, which is collected in an area 1130 below the second separator and transferred along pathway 1018 via a pump 1132 and a chiller 1134 for return back to the rotary screen 1102 for reuse as a rinse agent via wash entry points 1112. In some examples, as shown here, a portion of the cooling agent may be returned to the vessel (not shown) via pipeline 1136 through the horizontal axle 1104, which in this example is hollow and provided with rinse points 1138. As previously noted, the first separator's 1008 output, cooling agent containing entrained trichomes and fine particulate matter via the collection area 1124 and pathway 1010, and the mostly spent cannabis plant material via 1014, substantially matches the input of fragments and particulate matter, from the vessel, into the upstream end 1106 of the screen. This allows a continuous throughput for the system when in operation.

The present method, system and apparatus is advantageous as it does away with the prolonged drying period that follows harvesting of the cultivated cannabis plants and which is required in conventional techniques. Instead, the plant material may be processed relatively quickly following its harvesting, with minimal drying or dehumidification required as a pre-processing stop. In addition, once pre-processing has commenced, the use of a number of processing modules in series allows for continuous processing of harvested cannabis plant material. Alternatively, a continuous processing module as described with reference to FIGS. 10 and 11, is able to process a continuous feed of cannabis plant material further improving efficiencies.

The systems and methods described herein may also reduce processing times in a cost-effective manner, reduce solvent volumes required to process plant material, improve extraction efficiencies of value components from natural plant material.

The forgoing advantages help maximise the extraction of the valuable components of the processed plant material, while at the same minimising wastage and potential labour costs. The use of multiple separators to work the harvested cannabis plant material into high and low value streams may enhance efficiencies of operation.

Unless the context clearly requires otherwise, through the description and the claims, the words “comprise”, “comprising”, and the like are to be construed in an inclusive sense as opposed to an exclusive or exhaustive sense, that is to say, in the sense of “including, but not limited to”.

Wherein the foregoing description reference has been made to integers or components having known equivalents thereof, those integers are herein incorporated as if individually set forth.

The disclosure may also be said broadly to consist in the parts, elements, characteristics and features referred to or indicated in the specification of the application, individually or collectively, in any or all combinations of two or more of said parts, elements, characteristics or features.

Aspects of the present disclosure have been described by way of example only and it should be appreciated that modifications and additions may be made thereto without departing from the scope thereof as defined herein.

It should be noted that various changes and modifications may be made without departing from the spirit and scope of the disclosure as claimed and without diminishing its attendant advantages. It is therefore intended that such changes and modifications be included within the present claims.

Reference to any prior art in this specification is not, and should not be taken as, an acknowledgement or any form of suggestion that the prior art referred to forms part of the common general knowledge in the field of endeavour in any country in the world. The disclosure of any applications, patents and publications cited herein, if any, are herein incorporated by reference.

1-72. (canceled)

73. A system for use in a method of separating trichomes from harvested cannabis plant material, wherein the system comprises:
a processing module that comprises:
a vessel, wherein the vessel is configured to receive a cooling agent and harvested cannabis plant material and wherein the vessel is provided with an outlet;
an agitator configured to agitate the harvested cannabis plant material in the vessel;
a first separator and a second separator;
the processing module comprising a first flow path from the outlet of the vessel to the first separator, the flow path arranged to transfer a laden quantity of cooling agent containing a filtrate of agitated harvested cannabis plant material comprising at least some trichomes to the first separator; and
a second flow path from the first separator to the second separator, the second flow path arranged to transfer a first stream of material derived from the filtrate from the first separator to the second separator.

74. The system as claimed in claim 73, wherein the processing module comprises a filtering container, wherein the filtering container is configured to be received within the vessel and receive harvested cannabis plant material.

75. The system as claimed in claim 74, wherein the filtering container is a substantially cylindrical basket with a continuous side wall comprising a plurality of perforations.

76. The system as claimed in claim 73, wherein the dimensions of the filtering container are 80% to 95% of the dimensions of the vessel.

77. The system as claimed in claim 73, wherein the processing module comprises a charger which is configured to introduce the cooling agent to the vessel.

78. The system as claimed in claim 73, wherein the processing module comprises a charger for the harvested cannabis plant material which is configured to introduce the harvested cannabis plant material to the vessel.

79. The system as claimed in claim 73, wherein the processing module comprises a mill to reduce the size of the harvested cannabis plant material prior to introducing it to the vessel.

80. The system as claimed in claim 73, wherein the agitator is configured to aggressively mechanically work and break up the harvested cannabis plant material into shreds and/or particulate matter and work it into a mixture or slurry with the cooling agent.

81. The system as claimed in claim 73, wherein the first and second flow paths include a pump.

82. The system as claimed in claim 81, wherein the pump is configured to aggressively agitate the filtrate prior to being introduced to the first separator.

83. The system as claimed in claim 73, wherein the processing module is configured to deliver an amount of cooling agent and harvested cannabis plant material to the vessel in an amount and at a rate substantially the same as the amount and rate at which the filtrate is delivered to the first and/or second separator.

84. The system as claimed in claim 73, wherein the first separator comprises a rotating screen having a first end and second end.

85. The system as claimed in claim 84, wherein the filtrate is delivered to the rotating screen at the first end.

86. The system as claimed in claim 84, wherein the first separator comprises a conveyor at or proximate the second end of the rotating screen, the conveyor receiving the filtrate after it has passed through the rotating screen.

87. The system as claimed in claim 86, wherein the conveyor is a helical conveyor.

88. The system as claimed in claim 86, wherein the conveyor is inclined and has an upper end and a lower end.

89. The system as claimed in claim 95, wherein the conveyor has an upper outlet at its upper end and a lower outlet at or proximate its lower end.

90. The system as claimed in claim 89, wherein the upper outlet of the conveyor is an exit point for harvested cannabis plant material separated from the filtrate passing through the conveyor.

91. The system as claimed in claim 89, wherein the lower outlet of the conveyor is an exit point for a first stream of material derived from the filtrate.

92. The system as claimed in claim 89, wherein the lower outlet of the conveyor is communicative with the flow path to the second separator.

93. The system as claimed in claim 84, wherein the first separator comprises one or more rinse down points proximate the rotating screen.

94. The system as claimed in claim 93, wherein the first separator comprises a flow path from a screen receiving the rinse agent to the second separator.

95. The system as claimed in claim 93, wherein the second separator comprises a first flow path for the trichomes derived from the filtrate and a second flow path for cooling agent derived from the filtrate.

96. The system as claimed in claim 95, wherein the second flow path leads to the rinse down points for the first separator.

97. The system as claimed in claim 95, wherein the second flow path returns to the vessel of the processing module.

98. The system as claimed in claim 73, wherein the second separator is a rotary drum screen.

99. A method of using a system as claimed in claim 73, the method comprising the steps of:
a) filling the vessel with a cooling agent and harvested cannabis plant material;
b) agitating the mixture of harvested cannabis plant material and cooling agent to form a laden quantity of cooling agent containing a filtrate of agitated harvested cannabis plant material comprising at least some trichomes;
c) transferring a quantity of the filtrate to the first separator;
d) separating at least a portion of the filtrate into a first stream containing trichomes and cooling agent and a second stream containing harvested cannabis plant material; and
e) transferring the first stream to the second separator.

100. The method as claimed in claim 99, wherein the method comprises an additional step of:
f) using the second separator to separator the first stream into a high value stream of trichomes and a stream of spent cooling agent.

101. The method as claimed in claim 99, wherein during step a), the harvested cannabis plant material is placed into a filtering container which in turn is placed into the vessel.

102. The method as claimed in claim 99, wherein during step a) the cooling agent and harvested cannabis plant material is added to the vessel in an amount and at a rate substantially corresponding to the amount and rate at which the filtrate is transferred to the first separator during step c).

103. The method as claimed in claim 99, wherein during step b) the agitator aggressively mechanically works and breaks down the harvested cannabis plant material into shredded and particulate plant matter and trichomes.

104. The method as claimed in claim 99, wherein during step b) the shredded cannabis plant material and trichome are worked into a mixture or slurry with the cooling agent.

105. The method as claimed in claim 99, wherein during step c), at least some of the harvested cannabis plant material is filtered and prevented from being transferred to the first separator.

106. The method as claimed in claim 99, wherein during step d), the filtrate is rinsed by a rinse agent as it passes through the first separator.

107. The method as claimed in claim 106, wherein at least some of the rinse agent is cooling agent reclaimed from the first stream passing through the second separator during step f).

108. The method as claimed in claim 99, wherein the method comprises the step of collecting the rinse agent, and more preferably separating any trichomes contained therein from the rinse agent.

109. The method as claimed in claim 99, wherein the method comprises the step of milling the harvested cannabis plant material prior to introducing same to the vessel.
